(19) United States
(12) Patent Application Publication
(10) Pub. No.: US 2007/0224175 A1
(43) Pub. Date: Sep. 27, 2007

(54) Salt and pH tunable resist patterns on polyelectrolyte multilayers

(76) Inventors: Christina Chan, Okemos, MI (US); Ilsoon Lee, Okemos, MI (US); Srivatsan Kidambi, Cambridge, MA (US)

Correspondence Address: HARNESS, DICKEY & PIERCE, P.L.C., P.O. BOX 828, BLOOMFIELD HILLS, MI, 48303, US

(21) Appl. No.: 11/728,712

(22) Filed: Mar. 27, 2007

Related U.S. Application Data
(60) Provisional application No. 60/786,286, filed on Mar. 27, 2006.

(51) Int. Cl. A61K 35/12; C12N 5/06; C12N 5/08; A61K 9/70

(52) U.S. Cl. 424/93.7; 424/443; 435/325; 435/366; 427/2.24

(57) ABSTRACT

A method for selectively removing a resist material from the polycationic surface of a polyelectrolyte multilayer (PEM) film, without disturbing adhering interactions between the cationic film surface and bound biomaterials such as cells, proteins, and nucleic acids. The resist material is one that that inhibits or prevents the further deposition of cells or other biomaterial; it thus masks the cationic surface from application of biomaterials. In one embodiment the resist material is a carboxy functional oxyalkylene oligomer. It is removed by exposing the film containing the bound biomaterial and the bound resist material to a pH below 4.5, and/or to a salt concentration of higher than 0.01 M.

### CROSS REFERENCE TO RELATED APPLICATIONS

This application claims the benefit of U.S. provisional application 60/786,286 filed Mar. 27, 2006, the full disclosure of which is incorporated by reference.

### GOVERNMENT SUPPORT

The subject matter described herein was developed in part with funds from the National Science Foundation under contracts BES No. 0222747, No. 0331297, and No. 0425821 and CTS 0609164, and under funding by the Environmental Protection Agency (RD83184701), AFOSR (FA9550-06-1-0417), and the National Institute of Health (1R01GM079688-01). The U.S. government has certain rights in the invention.

### INTRODUCTION

Over the past decades, the development of new methods for fabricating thin films that provide precise control of the three-dimensional topography and cell adhesion has generated lots of interest. These films could lead to significant advances in the fields of tissue engineering, drug delivery and biosensors which have become increasingly germane applications in the field of chemical engineering.

The ionic layer-by-layer (LBL) assembly technique, introduced by Decher in 1991 has emerged as a versatile and inexpensive method of constructing polymeric thin films, with nanometer-scale control of ionized species. Films formed by electrostatic interactions between oppositely charged poly-ion species to create alternating layers of sequentially adsorbed poly-ions are called “Polyelectrolyte Multilayers (PEMs)”. PEMs have long been utilized in such applications as sensors, electrochromics, and nanomechanical thin films but more recently they have also been shown to be excellent candidates for biomaterial applications due to 1) their biocompatibility and bioinertness 2) the ability to incorporate biological molecules, such as proteins and 3) the high degree of molecular control of the film structure and thickness providing a much simpler approach to construct complex 3D surfaces as compared with photolithography.

Poly(ethylene glycol) (PEG) and its oligomeric derivatives have been used to resist nonspecific adsorption of polyelectrolytes, charged particles and proteins onto surfaces from aqueous solution. New surfaces for tissue engineering applications which have the capability of building 3D complex structures to control cell microenvironment is of major interest among the research community.

The ability to control cell adhesion in vitro has application to diverse fields, ranging from cell biology to tissue engineering. A number of fabrication strategies such as photolithography, microcontact printing, micromolding, inkjet printing and dip-pen spotting, have been applied to create micropatterned surfaces for manipulating the cell environment. In these approaches, the cells have been localized to adhesive regions on a substrate, thus limiting their use to one cell type. Most cell patterning studies that engineered patterned co-cultures have involved selective adhesion of one cell type over another. For example, studies to design co-cultures with primary hepatocytes and fibroblasts requires the adhesion of primary hepatocytes first before attaching the second cell type due to non-availability of an universal resist surface for all cells. Similarly, to design co-cultures with primary neuronal and astrocytes requires the adhesion of primary neuronal cells first before attaching the second cell type.

Protein immobilization in micron and nano-scale patterns has importance for drug discovery and delivery, bioengineering and biosensors, and fundamental studies of cell biology. Several techniques such as photolithography, soft lithography, photochemical methods, and dip-pen nanolithography have been developed, but have primarily focused on immobilizing one protein in defined regions surrounded by a “background” that lacks protein (and may be additionally resistant to the adsorption of other proteins from solution). To mimic complex cell-cell and cell-extracellular matrix interactions for studying problems in immunology, surfaces comprising of regions of multiple functional protein at cellular and subcellular length scales would be useful. However, few methods have been reported that allow patterning of multiple proteins on surfaces, and they often have limitations in spatial resolution, or in patterning fragile proteins due to their inability to withstand dehydration or exposure to organic solvents.

### SUMMARY

Self assembled monolayer patterns of resist material such as carboxy functional oxyalkylene oligomers (exemplified by m-d-poly(ethylene glycol) (m-dPEG)) are formed onto PEMs and subsequently removed from the PEM surface by treating with salt or low pH solutions. In various embodiments, the patterned SAMs on PEMs are created by ionic interactions using a microcontact printing (μCP) technique. Resist patterns are formed atop the PEM films and inhibit or prevent further deposits of polymer (polyelectrolyte) layers, as well as resisting further deposits of biomaterials such as cells, proteins, and nucleic acids.

The resist material patterns are removable from the PEM surface at certain pH and salt conditions without affecting the PEM films underneath the SAMs or the adherence of various biomaterials bound to the surface.

The resist patterns can be removed using conditions that do not compromise the underlying polymers, charged particles and biological molecules, including living cells, deposited on the surface prior to the salt treatment. The removable surfaces can be used to form patterns of multiple biomaterials or particles. These salt and pH responsive PEG SAMs are useful in diverse applications—e.g. 1) optical technologies such as electroluminescent and conducting surfaces by using this as a template to make two component particle arrays on PEMs, and 2) biotechnology such as controlled microenvironment for cells and drug-delivery systems.

In one aspect, highly customizable PEM thin films are used to engineer in vitro cellular microenvironments using microfabrication techniques to control cell adhesion and for drug delivery applications. Removable surfaces provide a template for designing multiple regions of different protein, which is useful in immunology where complex cell-cell, cell-extracellular matrix interactions play important roles. The approach avoids exposing the proteins to conditions outside the narrow range of physiological pH, ionic strength and temperature and thus maintains the stability of the proteins. It provides an environmentally friendly and biocompatible route to designing versatile salt tunable surfaces. The template can be used to form arrays of nucleic acids, proteins and other biological molecules which have applications as biosensors and basic biological studies.

Further areas of applicability will become apparent from the description provided herein. It should be understood that the description and specific examples are intended for purposes of illustration only and are not intended to limit the scope of the present disclosure.

### DETAILED DESCRIPTION

The following description is merely exemplary in nature and is not intended to limit the present disclosure, application, or uses.

All papers referred to in the body of the description are hereby incorporated by reference.

In various aspects, the present disclosure is based on discovery of a method for selectively removing a resist material from the cationic surface of a polyelectrolyte multilayer (PEM) film, without disturbing adhering interactions between the cationic film surface and bound biomaterials such as cells, proteins, and nucleic acids. The resist material is one that that inhibits or prevents the further deposition of cells or other biomaterial; it thus masks the cationic surface from application of biomaterials. In one embodiment the resist material is a carboxy functional oxyalkylene oligomer. The method comprises exposing the film containing the bound biomaterial and the bound resist material to a pH below 4.5, and/or to a salt concentration of higher than 0.01 M, preferably greater than 0.1 M.

Thus, the salt form of the resist material is removed from the surface at a pH where the resist material in solution is at least partially in a protonated state. In various embodiments, removal is carried out at lower pH, but not so low as to have a harmful biological effect on the biomaterials. Exemplary pH runs from about 1 to about 4.5, for example from 2 to 4.5. Alternatively or in addition, the resist material is removed by a salting out procedure to obtain a kind of ion exchange that replaces the anionic resist material on the cationic surface of the PEM film. Faster kinetics and equilibrium for the removal are favored by higher salt concentrations, keeping in mind the salt concentration should not be so high as to have a deleterious biological effect on the bound biomaterials. Suitable salt concentrations have been found in the range above 0.01 M, for example in the range of 0.02 M to 1 M, or from 0.25 M to 0.5 M. In various embodiments, the nature of the salt is not particularly limited, as long as it has no deleterious biological effect. An exemplary salt is sodium chloride.

Because the resist material adheres to the cationic surface and thereby masks portions of the PEM film and blocks or inhibits deposition of material onto the masked portions of the film, it can be used in a sequence of masking, binding, and removing steps as described herein to prepare substrates having a wide variety of patterns of biomaterials or colloidal particles applied in patterns corresponding to any predetermined scheme. Thus, substrates are provided containing one, two, three, or more different cells, proteins, nucleic acids, or other biomaterials, arranged in patterns that can be used in a wide variety of applications.

In another aspect, a cationic resist material binds to a polyanionic layer of a PEM, and is salted off or removed by adjusting the pH to a value where it is no longer charged. Thus the cationic resist material is removable generally by raising the pH sufficient to neutralize the charge. As with the anionic resist material, it is desirable to carrying out the steps of the method avoiding extremes of pH that would tend to harm the biomaterials used in the method.

In one embodiment, a method of producing a two- or multi-dimensional array of biomaterial attached to a solid substrate is provided. The method comprises applying a resist material onto the outer layer of a polyelectrolyte multilayer (PEM) film in a pattern to form a masked substrate with masked and unmasked portions; in one aspect the outer layer of the PEM film is made of a polycation so that the unmasked portion is cationic, while in another the outer layer of the PEM is made of a polyanion so that the unmasked portion is anionic. Next, the masked substrate is exposed to a first biomaterial (or alternatively a colloidal particle), which results in the biomaterial adhering to the unmasked portions (i.e. the portions of the cationic or anionic PEM surface not covered by the resist material. Then the resist material is removed by exposing the substrate to a low pH (in the case of an anionic resist material bound to a polycationic PEM surface) or a high pH (in the case of a cationic resist material bound to a polyanionic PEM surface), and/or to a high salt concentration sufficient to remove the resist material but not the deposited first biomaterial or colloidal particle. Then the substrate can be exposed to a second biomaterial, which adheres to portions of the substrate unmasked in the previous step. When two biomaterials are deposited in this way, a two dimensional array of biomaterial is formed. Alternatively, multi-dimensional arrays (i.e. those containing three, four, or more different biomaterials) can be provided. Instead of applying the second biomaterial to all of the unexposed portions of the substrate as described above, the unexposed portions are first partially masked by application of the resist material in a pattern that only partly covers the unexposed portions. Then a second biomaterial is applied, adhering as before to those portions of the substrate that are exposed, meaning the exposed portions are covered neither with the first biomaterial nor with the resist material. After the second biomaterial is applied, the resist material is again removed as before. The process of the masking, exposing to biomaterial, and unmasking can be repeated as desired to provide a substrate with any desired pattern of biomaterial. In this way, two- or multidimensional arrays of cell, proteins, nucleic acids, other biomaterials, or colloidal particles can be made.

In a particular embodiment, a method of making a multidimensional array comprises, applying a second resist layer onto the substrate that only partly covers the unexposed portions that result from removing the resist material after applying the first biomaterial. This provides a substrate comprising a polycationic surface partly covered with the first biomaterial and partly covered with the (second) resist material. It is understood that in various embodiments the first and second (or subsequent) resist materials are made of the same chemical species—the terms “first”, “second”, and so forth designate the order in which they are applied. After subsequently exposing the substrate to the second biomaterial, the resist material is again removed to provide a substrate partly covered with the first biomaterial and partly covered with the second biomaterial, and further comprising exposed polycationic surface. Then a third biomaterial can be applied to the exposed portions of the substrate. Further biomaterials can be applied as desired in similar fashion.

In various embodiments, the resist material is applied by microcontact printing. Thus, in one embodiment, a method of making the arrays includes providing a polyelectrolyte multilayer (PEM) film comprising alternating layers of polycation and polyanion deposited on a solid substrate, wherein the outer layer of the film comprises a polycation. The outer layer is referred to equivalently herein as “cationic” or “polycationic”. Then a solution of a carboxy functional oxyalkylene oligomer is microcontact printed onto the outer layer of the PEM film in a masking pattern to produce a substrate with masked and unmasked portions. The masked portions are those areas where the resist material binds; the unmasked portions are the areas where no resist material is bound. As noted herein, the resist material attaches or binds to the outer cationic layer of the PEM in the so-called “self-assembled monolayers” (“SAMs”) described herein. Alternatively, microcontact printing is used to apply a cationic resist material onto a polyanionic outer layer of a PEM film.

A first component of cells is applied onto the unmasked portions of the substrate. The oligomer is then removed from contact with the masked portions by exposing the substrate to a low pH (or high pH, as discussed above) or high ionic strength. This uncovers or exposes the portions previously masked by the oligomer. In a subsequent step at least one second component of cells is applied onto the substrate, whereby the cells of the at least one second component adhere on unmasked portions of the surface.

Polyelectrolyte Multilayers

Films formed by electrostatic interactions between oppositely charged poly-ion species to create alternating layers of sequentially adsorbed poly-ions are called “polyelectrolyte multilayers” (PEM). Polyelectrolyte multilayers are prepared layer-by-layer by sequentially immersing a substrate, such as a silicon, glass, or plastic slide, in positively and then negatively charged polyelectrolyte solutions in a cyclic procedure. The process is described for example in Decher et al. Science (1997) 277, 1232-1237 and Decher et al., Makromol. Chem. Macromol. Symp. 1991, 46, 321-327, the disclosures of which are incorporated herein by reference. A wide range of negatively charged and positively charged polymers is suitable for making the layered materials. Suitable polymers are water soluble and sufficiently charged (by virtue of the chemical structure and/or the pH state of the solutions) to form a stable electrostatic assembly of electrically charged polymers. Sulfonated polymers are commonly used as the negatively charged polyelectrolyte. Non-limiting examples include sulfonated polystyrene (SPS); poly (anetholesulfonic acid) (PAS); and poly (vinylsulfonic acid) (PVS). Quaternary nitrogen-containing polymers such as poly (diallyldimethylammonium chloride) (PDAC) are commonly used as the positively charged electrolyte.

Assembly of the PEM's is well known. The method can be conveniently automated with robots and the like. Although a polyelectrolyte of either positive or negative charge can be applied first to the substrate, the process will be illustrated with an initial positive charge. In a non-limiting embodiment, a polycation is first applied to a substrate followed by a rinse step. Then the substrate is dipped into a negatively charged polyelectrolyte solution for deposition of the polyanion, followed again by a rinse step. The procedure is repeated as desired until a number of layers is built up. A bilayer consists of a layer of polycation and a layer of polyanion. Thus for example, 10 bilayers contain 20 layers, while 10.5 bilayers contain 21 layers. With an integer number of bilayers, the top surface of the PEM has the same charge as the substrate. With a half bi-layer (e.g. 10.5 illustrated) the top surface of the PEM is oppositely charged to the substrate. In this way, a PEM is built having either a negative or a positive charged polymer as the “top” or “outside” layer to which biological materials are attached. In various embodiments, such biological materials, particles, and resist materials are applied to a cationic (also called “polycationic”) outside surface of a PEM or to an anionic (also called “polyanionic”) outside surface.

Resist Material

The resist material used to form resistive patterns on the PEM's and provide for directed deposition of materials on uncovered or exposed portions of the surface is one that is soluble in water or a mixture of water and alcohol, binds effectively to a cationic or anionic surface of a PEM, and can be removed with the pH and/or salting out methods described herein. In various embodiments, for use when the outside surface of the PEM is cationic, the resist material is anionic, meaning it carries a functional group that is negatively charged at the use pH. An example is a carboxyl group, so the resist material is carboxy functional. In other embodiments, where the outside surface of the PEM is anionic, the resist material is cationic, meaning it carries a positive charge at the pH of use. An example is a protonated amine, so that the resist material is quaternary ammonium functional. The resist material is provided as a solution in water or other suitable solvents, such as alcohols and mixtures of water and alcohol, and applied onto the ionic surface of the PEM. Ionic and polar interaction of the PEM with biological materials such as cells, proteins, nucleic acids and the like is prevented at the areas of the surface where the resist material is bound. The surface thus resists further deposits of consecutive polymers, colloidal particles, nucleic acids, proteins and other macromolecules.

Structurally, the resist material contains two ends connected by a middle. The first end of the resist material is the one that binds electrostatically to the topmost layer of the PEM. In one aspect, the first end contains an anionic group that interacts with the cationic top layer of the PEM and that advantageously protonates at low pH. Suitable anionic groups include carboxylates. When the anionic group is protonated at low pH, the binding affinity is reduced because of the loss of ionic interactions and the resist material is removed from the surface. In another aspect, the first end contains a cationic group that interacts with an anionic surface of the PEM and that advantageously deprotonates at high pH. A non-limiting example is a quaternary ammonium group. When the quaternary ammonium group is deprotonated at low pH, the binding affinity is likewise reduced and the resist material is removed from the surface. Alternatively, at high salt concentrations, the cationic or anionic resist material is displaced from the PEM surface by a kind of mass balance or ion exchange phenomenon.

The second end of the PEG contains functional groups that inhibit or prevent binding of subsequent polycations, polyanions, proteins, nucleic acids, or other biological materials such as cells. The second end is essentially free of ionic groups or polar groups that would attract such biomolecules. Thus, in various embodiments the second end contains hydrocarbon or ether groups that are essentially non-polar. For example, a methyl ether group is conveniently provided by capping the terminal hydroxyl group of a polyoxyalkylene chain.

The middle of the resist material connects the two ends. In the case of protonated or quaternary fatty amines, or in the case of fatty soaps, the middle is hydrocarbon. In another exemplary embodiment, the middle is an oligomeric section of polyalkylene oxide (normally 2-20, preferably 2-10 oxides) of such a composition that the overall molecule is water soluble. For this reason it is referred to as an oxyalkylene oligomer. In various embodiments, the middle section has the structure of polyethylene oxide. The polyethylene oxide is also known as polyethylene glycol, or PEG. For this reason, the term “PEG” is used herein as a shortcut for this kind of structure (the oxyalkylene oligomer), even if some of the middle contains repeating units from propylene oxide or higher oxides. Further aspects are illustrated using PEG for convenience of reference, but the embodiments described in that way are not limited to using just the PEG type of resist material.

A representative PEG molecule useful in the invention is m-dPEG, sold by Quanta Biodesign. It has a structure 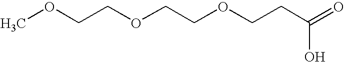
As drawn, the resist material has a carboxy functional first end, a second end that is an ether, and a middle section that is an oxyalkylene oligomer. As drawn, the oligomer is made of two repeating units of ethylene oxide.

In various embodiments, patterns of resist material are produced or laid down on the top cationic surface of a PEM as described herein. The resist molecule, by virtue of its ionizable anionic group on one end and non-polar group on the other end, binds to the surface, and prevents further binding of biomaterials and colloids to the surface. In an illustrative case of an anionic resist material, the activated carboxyl functional group ionically binds to the topmost positive (cationic) surface of the PEM and the other end resists the deposit of subsequent material. This enables the development of complex surface structures and templates for selective layer-by-layer deposition. As such, it is a kind of masking material. In this way PEG patterns such as those of m-dPEG acid on PEMs act as resistive templates.

In one embodiment, subsequent layers of polyanion and polycation can be added onto areas of the PEM not covered by the resistive layer. 3-D SAM's can be built in this way. In other embodiments, proteins, nucleic acids, cells, and the like are adsorbed onto areas of the PEM surface not covered by the resist material, to build up or form arrays of biomaterial or colloidal particles on a PEM coated substrate.

Applying the Resist Material—Microcontact Printing

The resist material is applied as a solution in a suitable solvent such as water or mixtures of water and alcohol, such as water and ethanol. Any method can be used to apply the resist material in patterns as required for the particular application. In various embodiments, the PEG patterns on PEMs are created by ionic interactions using a microcontact printing (μCP) technique. Microcontact printing was introduced by Whiteside's and co-workers (see Appl. Phys. Lett. 1993, 63, 2002-2004 and Angew. Chem. Int. Ed. 1998, 37, 550-575, the disclosures of which are incorporated by reference). The technique is also described in Kidambi et al., Journal of the American Chemical Society, Vol. 126, and pages 4697-4703 (2004), the entire disclosure of which is hereby incorporated by reference. It provides a versatile method of chemically and molecularly patterning surfaces at the submicrometer scale. It is characterized by high fidelity and ease of duplication. μCP uses an elastomeric stamp to print a variety of molecules with submicrometer resolution and without the need for dust-free environments and harsh chemical treatments. The stamp is coated with the desired molecules, and the molecules residing on the raised regions of the stamp are brought in contact with the host substrate when the stamp is printed. The resistive patterns of PEG can be removed according to the invention under conditions that do not adversely affect the properties of bound biomolecules. As a result, there can be achieved a directed deposition of a variety of macromolecules.

Preparation of Arrays of Biomolecules or Particles on Substrates

By using the patterned resist material, it is possible to direct the formation of bound biomaterials or colloidal particles to areas or portions of the PEM surface not previously covered with material and not masked with bound resist material. Such a directed assembly is carried out on a PEM surface by first depositing a resist material (such as a PEG molecule layer) in a first desired pattern, followed by subsequent attachment of protein, nucleic acid, cell, colloidal particle, and so on to the exposed (i.e. uncovered by the resist material) surface of the PEM. Following the first attachment step, the resist pattern (such as a PEG SAM) is removed by exposing it to high salt concentrations and/or low or high pH (depending on the anionic or cationic nature of the resist material), generating fresh active surface by removal of the resist material. Then, a second material is attached to the fresh active surface, to deposit the second material at positions originally masked by the PEG. In this way patterns of cells, proteins, nucleic acids, and the like are built up on PEM's. Alternatively, after the first attachment of protein, cells, nucleic acids and the like, a second PEG pattern is laid down of the surface of the PEM, where PEG is re-applied to some but not all of the areas originally not covered by the PEG. After subsequent addition of protein, nucleic acids, etc., the PEG resist layer can once again be removed to expose a fresh active surface. The process can be repeated to provide areal coverage of 2, 3, or more different proteins, nucleic acids, and cells, etc.

In various embodiments, 3-dimensional structures are provided on PEM's by building up SAM's of polyelectrolyte layers on surfaces not masked by a patterned deposition of a PEG resistive layer. Directed deposition of multiple proteins, nucleic acids, cells, and the like is carried out in a similar fashion on the 3-dimensional PEM SAM's. In various aspects, the removal conditions have been found not to adversely affect the binding or the biological activity of the proteins, nucleic acids, cells, and the like.

Thus it is seen that the removable surfaces of PEG provide a template for designing multiple regions for example of different protein, which is useful in immunology where complex cell-cell or cell-extracellular matrix interactions play important roles. The PEG patterns can be removed using conditions that do not compromise the underlying polymers, charged particles, and biological materials, including living cells, deposited on the surface prior to the salt treatment. In non-limiting embodiments, the removable surfaces can be used to form patterns of multiple proteins and cells.

Stamping of m-dPEG Acid

Various factors are evaluated in determining suitable conditions for the stamping process. These include plasma treatment or not of the PDMS stamps, the type and concentration of the solvents used in making the ink solution, the pH of the solution, and the contact times. In various embodiments, PDMS stamps are used that are not treated with oxygen plasma. In some cases it can be experimentally determined that untreated PDMS stamps result in more complete transfer of patterns when compared to the stamps treated with plasma. A solvent of 75% (v/v) ethanol/water has been found to give satisfactory results in terms of the effective transfer of the ink solution from the PDMS stamps onto the surface.

The stamps can be inked in various methods, including spin-inking, cotton swab-inking, and dip-inking. In various embodiments, the dip-inking method results in the most efficient transfer of the ink onto the PEM surface. The contact times can also be varied from a few seconds to 30 min. Suitable results are obtained with a 15 min contact time.

The pH of the inking solution also plays an important role. This is believed to be because the ionic group of the resist material is responsible for the ionic interaction between the resist material and the ionic surface of the PEM. The extent of ionization of the resist material affects the strength of the ionic bond between the two ions and thus the extent of the pattern transfer. The ionization of the resist material, in turn, depends on the pH of the solution. For anionic resist materials such as carboxyl functional ones, it is generally preferred to maintain the pH of the inking solution at about 4.5 or higher, such as 4.5 to 6. Likewise, for cationic resist materials, it is preferred to maintain the pH at a value where the resist material is positively charged.

The pH of the inking solution is one in which the ionic groups of the resist material are charged, whereas for removal the pH is adjusted to a range where the resist material is uncharged.

Biomaterials

Biomaterials include proteins, nucleic acids, and cells. Such materials have been found to bind to the outer cationic surface of a PEM prepared as described herein. In various embodiments, proteins include enzymes, antibodies, gene products, cell receptors, synthetic polypeptides, peptide oligomers, and so on. Nucleic acids include ribonucleic acids, deoxyribonucleic acid, and aptamers, including gene markers, expressed DNA and RNA fragments, synthetic nucleotides, and the like. Cells include prokaryotic and eukaryotic cells. Non-limiting examples of eukaryotic cells include primary hepatocytes, primary neurons, primary astrocytes, PC12 cells, SH-SY5Y cells, HeLa cells, and fibroblasts.

The biomaterials are applied to the substrates described herein in any suitable manner. For example, a solution or suspension of the biomaterial is brought into contact with the substrate for a sufficient time for the biomaterial to attach to the surface to provide the desired coverage. The time for coverage will vary according to conventional kinetic considerations such as temperature and concentration of the biomaterial. Suitable time, temperature and concentrations can be worked out in individual cases. To illustrate in a non-limiting embodiment, cell media are used that contain from about 10⁵ to 10⁷ cells per mL of suspension. A physiological temperature of about 37° C. is used in various embodiments. Conditions are further illustrated in the Examples below.

In one aspect, the disclosure describes methods of controlling cell adhesion. The methods provide the ability to dynamically and locally switch substrate adhesiveness to different types of cells, which facilitates the patterning of two or more cell types in spatially defined co-cultures. In various aspects, the removable resist material and methods described herein provide surfaces that can be readily switched from cell-repulsive to cell-adhesive using cell friendly conditions. This approach is advantageous since it can be used to form patterned co-cultures irrespective of the types of cell or the order of seeding of the different types of cells and exposes the cells to conditions within the physiological range of pH, ionic strength and temperature.

In another aspect, the methods provide a template for designing multiple regions of different proteins onto defined regions of a surface without exposing the proteins to irradiation, organic solvents or dehydration. An advantage of this approach is that it exposes the proteins to conditions within the narrow range of physiological pH, ionic strength and temperature where their stability is maintained.

### EXAMPLES

Experimental Materials

Sulfonated poly(styrene), sodium salt (SPS) (Mw=70000), poly(diallyldimethylammonium chloride) (PDAC) (Mw=100000-200000) as a 20 weight % solution, and sodium chloride were purchased from Aldrich Chemical, Milwaukee, Wis. The m-dPEG acid molecule (Mw=236) was obtained from Quanta Biodesign. Poly(dimethylsiloxane) (PDMS) from the Sylgard 184 silicone elastomer kit (Dow Corning, Midland, Mich.) was used to prepare stamps. The fluorosilanes was purchased from Aldrich Chemical. These PDMS stamps were used for microcontact printing. Glass slides (Corning Glass Works, Corning, N.Y.), used for making the polyelectrolyte multilayer films, were cleaned using a Bransonic ultrasonic cleaner (Bransonic Ultrasonic Corporation, Danbury, Conn.). Carboxyfluorescein (6-CF), fluorescence dye, was purchased and used as received from Sigma. Carboxylated polystyrene latex particles (4 μm diameter), purchased from Polysciences, were used for colloidal adsorption study on m-dPEG self-assembled monolayer patterned polyelectrolyte templates.

### Example 1

### Preparation of Polyelectrolyte Multilayers

In an exemplary embodiment, PDAC and SPS polymer solutions are prepared with deionized (DI) water at concentrations of 0.02M and 0.01 M, respectively, (based on the repeating unit molecular weight) with the addition of 0.1 M NaCl salt. A Carl Zeiss slide stainer equipped with a custom-designed ultrasonic bath was connected to a computer to perform layer-by-layer assembly. To form the first bilayer, the tissue culture polystyrene (TCPS) plates are immersed for 20 min in a polycation solution. Following two sets of 5 min rinses with agitation, the TCPS plates are then placed in a polyanion solution and allowed to deposit for 20 min. Afterwards, the 6 well plates are rinsed twice for 5 min each. This process is repeated to build multiple layers. All experiments are performed using ten (i.e., 20 layers) or ten and half bilayers (i.e., 21 layers).

### Example 2

### Preparation of PDMS Stamps

An elastomeric stamp is made by curing poly(dimethylsiloxane) (PDMS) on a microfabricated silicon master, which acts as a mold, to allow the surface topology of the stamp to form a negative replica of the master. The poly(dimethylsiloxane) (PDMS) stamps are made by pouring a 10:1 solution of elastomer and initiator over a prepared silicon master. The silicon master is pretreated with fluorosilanes to facilitate the removal of the PDMS stamps from the silicon masters. The mixture is allowed to cure overnight at 60° C.

### Example 3

### Characterization

A Nikon Eclipse ME 600 optical microscope (Nikon, Melville, N.Y.) is used to obtain dark field images of the m-dPEG acid patterns and the additional microfabricated PEMs. A Nikon Eclipse E 400 microscope is used to obtain fluorescence images. 6-carboxyfluorescein (6-CF) dye is used to visualize m-dPEG SAM patterns on PEM following the stamping and rinsing processes. The dye is dissolved directly in 0.1 M NaOH; samples are imaged by dipping the substrates into the dye solution. The dye, which is negatively charged, preferentially stains the positively charged PDAC surface. The dyed regions appear green when viewed with the fluorescence optical microscope, using an FITC filter. Images are captured with a digital camera and processed on a Pentium computer running camera software.

In experimental embodiments, polyanion or polycation (unmasked) surfaces of PEM's are visualized with dyes that fluoresce in the presence of the charged materials. Such techniques along with fluorescence tagging of various biological materials can also be used to demonstrate the patterned assembly made possible by using the methods of the invention. Immunological methods can also be used.

### Example 4

### Directed Assembly of Proteins

To show that the salt conditions do not adversely affect the biomolecules, two proteins are attached to a PEM, one before and one after PEG removal. Before salt treatment to remove the PEG resist layer, an Alexa Fluoro tagged protein is visualized by fluorescence on the non PEG region. After treating the surface with salt, which removes the PEG, an FITC tagged protein is added onto the new active surface. Both proteins can be seen on the surface as an array when observed through a fluorescence microscope.

### Example 5

### Directed Assembly of Proteins

The same protein secondary alcohol dehydrogenase (sADH) with different fluorescent tags (FITC and Alexa Fluoro), is attached onto the PEG patterns before and after salt treatment indicating that the salt treatment did not affect the proteins attached to the PEM surface. Fluorescence images of the directed assembly of proteins on top of PEG patterns show both red and green channels before salt treatment. The red regions demonstrate the directed attachment of the Alexa Fluoro tagged secondary alcohol dehydrogenase (sADH) proteins to the PEMs [(PDAC/SPS)_{10.5}] and away from the resistive m-dPEG monolayer regions (black regions). No proteins are observed when imaged using the green channel. When the protein-deposited surfaces are treated with salt, the proteins remain attached and intact while the PEG SAMs are removed exposing the underlying active PDAC surface. Next FITC-tagged sADH are deposited over the exposed surfaces resulting in a two protein array.

### Example 6

### Attachment of Cells

HeLa cells are attached onto the PEG patterns before and after salt treatment indicating that the salt treatment removes the resist material but does not affect the cells attached to the PEM surface. In this study, the same cell type is used to illustrate the method's ability to control the adhesion of similar cell types. This approach can be extended to different combinations of cell types for co-culturing (e.g., adherent versus non-adherent, eukaryotic versus prokaryotic). Phase contrast and fluorescence images are taken of the directed assembly of FITC tagged HeLa cells on top of the PEG patterns before salt treatment. When the cell-deposited surfaces are treated with salt, the cells remain attached and intact while the PEG SAMs (resist material) are removed exposing the underlying active PDAC surface. Next, a second batch of HeLa cells without the fluorescent tag is deposited over the exposed surfaces resulting in a patterned co-culture of HeLa cells. When the cells are imaged under the fluorescent microscope, the HeLa cells seeded before the salt treatment are visible while the cells added after the salt treatment are not observed due to the lack of fluorescent tag on the cells.

### Example 7

### Directed Assembly of Colloidal Particles

m-dPEG acid molecules are stamped on top of a (PDAC/SPS)_{10.5} PEM and negatively charged carboxylated polystyrene PS particles (Polysciences, Diameter=0.5 μm) deposited selectively over the positive (PDAC/SPS)_{10.5} surface but not on the m-dPEG self-assembled monolayer regions. When the particle-deposited surfaces are treated with salt, the particles remain attached and intact while the PEG SAMs (resist material) are removed exposing the underlying active PDAC surface. Next, particles of 0.2 μm diameter are deposited over the exposed surfaces resulting in a two particle system. The different sizes of the two particles can be readily visualized, showing a two dimensional array. This approach has potential applications in electronic and optical devices, direct colloid assembly, plastic electronics and thin film power devices.

### Example 8

### Hepatocyte Isolation

Primary rat hepatocytes were isolated from 2 months old adult female Sprague-Dawley rats (Charles River Laboratories, Boston, Mass.), according to a two-step collagenase perfusion technique described by Seglen in Methods in Cell Biology 13, 29-83 (1976) and modified by Dunn in Biotechnology Progress 7, 237-45: (1991).

The liver isolations yielded 150-300×10⁶ hepatocytes. Using trypan blue exclusion the viability ranged from 90 to 98%. Primary hepatocyte culture medium consisted of DMEM supplemented with 10% FBS, 14 ng/ml glucagon, 20 ng/ml epidermal growth factor, 7.5 μg/ml hydrocortisone, 200 μg/ml streptomycin (10,000 μg/ml)—penicillin (10,000 U/ml) solution, and 0.5 U/ml insulin.

### Example 9

### Hepatocyte Culture

The cells were seeded under sterile tissue culture hoods and maintained at 37° C. in a humidified air/CO₂ incubator (90/10 Vol %). Primary hepatocytes were cultured on PEM coated 6-well TCPS coating PEG patterns. The multilayer coated TCPS plates were sterilized by spraying with 70% ethanol and exposing them to UV light before seeding the cells onto these surfaces. The cell culture experiments were performed on PEM surfaces without adhesive proteins. Collagen coated TCPS and uncoated TCPS were used as controls in these studies. A collagen gel solution was prepared by mixing 9 parts of the 1.2 mg/ml collagen suspension in 1 mM HCl with 1 part of concentrated (10×) DMEM at 4° C. The control wells were coated with 0.5 ml of this collagen gel solution and the coated plates were incubated at 37° C. for 1 hour. Freshly isolated hepatocytes were seeded at a concentration of 2×10⁵ cells per well and 2 mL were added to all the surfaces studied. One mL of fresh medium was supplied daily to the cultures after removal of the supernatant. Samples were kept in a temperature and humidity controlled incubator.

### Example 10

### NIH 3T3, HeLa Cell Culture

NIH 3T3 fibroblast and HeLa cell lines were purchased from American Tissue Type Collection. Cells grown to 70% confluency were trypsinized in 0.01% trypsin (ICN Biomedicals) solution in PBS for 10 min and re-suspended in 25 mL media. Approximately 10% of the cells were seeded into a fresh tissue culture flask and the rest of the cells were used for the co-culture experiments. Fibroblast medium consisted of DMEM with high glucose, supplemented with 10% bovine calf serum and 200 U/mL penicillin and 200 μg/mL streptomycin.

### Example 11

The removable PEG SAMs developed in this study provide surfaces that can be readily switched from cell-repulsive to cell-adhesive using cell friendly conditions. This approach is advantageous since it can be used to form patterned co-cultures irrespective of the types of cell or the order of seeding of the different types of cells. The first cell type is attached onto the PEG patterns before salt; after salt treatment (250 mM) the PEG is removed and the second cell type is seeded on the newly exposed surface. The procedure for seeding the cells is described in brief, as follows: Substrates with PEM and PEG patterns are sterilized under UV light overnight. Primary hepatocytes are seeded onto the PEG patterns at a cell density of 1.0×10⁶ per mL in serum-free media for 36 h at 37° C., 10% CO₂, balance air. On the hepatocyte-containing substrates, salt is then added for a period (for example, 1 hour) after which the salt media is removed and NIH 3T3 cells are seeded at a density of 0.5×10⁶ cells/well and incubated in primary hepatocyte media at 37° C. The fibroblast/hepatocyte ratio used in this study is 0.5:1 which is the approximate physiologic ratio of stromal:parenchymal cells in the liver.

We claim:

1. A method of producing a two- or multi-dimensional array of biomaterial attached to a solid substrate, comprising
applying a resist material onto the outer layer of a polyelectrolyte multilayer (PEM) film in a pattern to form a masked substrate with masked and unmasked portions, wherein the outer layer comprises a polycation or a polyanion;
exposing the masked substrate to a first biomaterial whereby the biomaterial adheres to the unmasked portions;
exposing the substrate to a pH or high salt concentration sufficient to remove the resist material but not the first biomaterial; and then
exposing the substrate to a second biomaterial, whereby the second biomaterial adheres to portions of the substrate unmasked in the previous step to form a two dimensional array of biomaterial.

2. A method according to claim 1, wherein the first and second biomaterials are cells.

3. A method according to claim 1, wherein the first and second biomaterials are proteins.

4. A method according to claim 1, wherein the first and second biomaterials are nucleic acids.

5. A method according to claim 1, wherein the resist material comprises a carboxy functional water soluble molecule.

6. A method according to claim 5, wherein the resist material comprises a polyoxyalkylene.

7. A method according to claim 1, wherein the resist material is a carboxy functional polyoxyalkylene in a solution at a pH at or above the pK of the carboxy functional group.

8. A method according to claim 1, whereby applying the resist material is accomplished by microcontact printing.

9. A method according to claim 1, wherein the outer surface of the PEM is a polycation.

10. A method according to claim 1, further comprising, prior to exposing the substrate to the second biomaterial,
applying a second resist layer onto the substrate that only partly covers the unexposed portions that result from removing the resist material, to provide a substrate comprising an ionic surface partly covered with the first biomaterial and partly covered with the resist material; and after subsequently exposing the substrate to the second biomaterial,
removing the resist material to provide a substrate partly covered with the first biomaterial and partly covered with the second biomaterial, and further comprising exposed ionic surface; and subsequently applying a third biomaterial to exposed portions of the substrate.

11. A method for preparing an array of cells attached to a surface in a predetermined pattern, the method comprising
providing a polyelectrolyte multilayer (PEM) film comprising alternating layers of polycation and polyanion deposited on a solid substrate, wherein the outer layer of the film comprises a polycation;
microcontact printing a solution of a carboxy functional oxyalkylene oligomer onto the outer layer of the PEM film in a masking pattern to produce a substrate with masked and unmasked portions;
applying a first component of cells onto the unmasked portions of the substrate;
removing the oligomer from contact with the masked portions by exposing the substrate to a low pH or high ionic strength, thereby exposing the portions previously masked by the oligomer; and in a subsequent step
applying at least one second component of cells onto the substrate, whereby the cells of the at least one second component adhere on unmasked portions of the surface.

12. A method according to claim 11, wherein the polycation of the outer layer is poly(diallyldimethyl ammonium chloride).

13. A method according to claim 11, wherein the polyanion is selected from poly (anethole sulfonic acid), sulfonated polystyrene, and poly (vinyl sulfonic acid).

14. A method according to claim 11, wherein the polyanion comprises sulfonated polystyrene.

15. A method according to claim 11, wherein the oligomer is m-dPEG.

16. A method according to claim 11, wherein the pH of the oligomer solution in the microcontact printing step is 4.5 or higher.

17. A method according to claim 11, wherein the oligomer is removed by exposing it to a pH below 4.5.

18. A method according to claim 11, wherein at least one of the cell components is selected from primary hepatocytes, HeLa cells, fibroblasts, primary astroyctes, PC12 cells, SH-SY5Y cells, and primary neurons.

19. A method according to claim 11, wherein at least one of the cell components comprises prokaryotic cells.

20. A method according to claim 11, wherein at least one of the cell components comprises eukaryotic cells.

21. A method for selectively removing a resist material from the polycationic surface of a polyelectrolyte multilayer (PEM) film, the film surface comprising bound cells and bound resist material that resists the further deposition of cells, the method comprising exposing the film containing the bound cells and the bound resist material to a pH below 4.5, and/or to a salt concentration of higher than 0.01 M.

22. A method according to claim 21, comprising exposing the film to a pH of 1.0 to 4.5.

23. A method according to claim 21, comprising exposing the film to a pH of 2 to 4.5.

24. A method according to claim 21, comprising exposing the film to a salt concentration of 0.01 M to 1.0 M.

25. A method according to claim 21, comprising exposing the film to a salt concentration of 0.1 M to 0.5 M.

26. A method according to claim 21, wherein the bound resist material is a carboxyl functional oxyalkylene oligomer.

27. A method according to claim 21, wherein the bound resist material is m-dPEG.
